(19) United States
(12) Patent Application Publication
(10) Pub. No.: US 2009/0062695 A1
(43) Pub. Date: Mar. 5, 2009

(54) APPARATUS FOR MEASURING AND ANALYZING EFFORTS EXERTED BY THE LEGS

(75) Inventors: Bernard Sauvignet, Roche La Moliere (FR); Antoine Gaucher, Andrezieux Boutheon (FR)

Correspondence Address: HESLIN ROTHENBERG FARLEY & MESITI PC, 5 COLUMBIA CIRCLE, ALBANY, NY, 12203, US

(73) Assignee: H.E.F., Andrezieux Boutheon (FR)

(21) Appl. No.: 11/577,582

(22) PCT Filed: Oct. 20, 2005
(86) PCT No.: PCT/FR05/50855

§ 371 Date: Apr. 19, 2007

(30) Foreign Application Priority Data
Oct. 20, 2004 (FR) 0452374

(51) Int. Cl. A61B 5/103

(52) U.S. Cl. 600/592

(57) ABSTRACT

The invention concerns methods and apparatus for measuring and analyzing leg effort. The apparatus includes a motorized treadmill secured to two superimposed upper and lower assemblies that are mounted in combination with measurement sensors. The apparatus may be used for analyzing the forces exerted while running, for example, by means of the lower assembly, or walking, by means of both the lower and upper assemblies.

The invention relates to the technical field of analysis and measurement apparatus for biomechanical medical use, medical and/or sports use in particular

Various types of apparatus, generally in the form of treadmills capable of measuring the ground reaction forces of the legs of an individual are known. Such measurements are very often necessary in order to analyse walking or running action.

The reader is reminded that walking is characterised by a double-support phase and that the right and left feet are simultaneously in contact with the ground during this phase. The feet follow two substantially parallel separate lines that can be approximately 15 to 30 cm apart. The measured forces are of the order of the value of the subject's weight.

Conversely, running is characterised by a so called “recovery” phase between each stride when the legs are not in contact with the treadmill. The forces involved are very high and can be as much as three times the weight of the subject as far as vertical forces are concerned. Contact between the feet and the treadmill is no longer in the form of two separate parallel lines and generates separate impacts.

Various technical solutions have been proposed in order to analyse walking or running.

As far as walking is concerned, one advantageous solution emerges from the teaching of Patent FR 2.730.154, the holder of which is the present Applicant. This patent concerns a device for measuring the forces exerted by each leg during walking and comprises two independent assemblies located alongside each other in parallel to match each of the legs. Each assembly is separately slaved to force measuring sensors supported by a common floor support plate. Each of the assemblies is equipped with a motor-driven treadmill mounted on a frame. The measuring sensors are chosen so as to measure the orthogonal components of a dynamic or quasi-static force acting in any direction.

This solution is especially suitable for analysing walking but is not suitable for analysing running. In fact, the two separate treadmills create an overhang and consequently a lack of rigidity required in order to analyse running action.

A single measurement platform is sufficient for running, given the fact that it is easy to separate the signals produced by the right foot and the left foot because the mediolateral forces are of opposite sign.

Various technical solutions have been suggested in order to measure the forces exerted during running. Essentially, the treadmill comprises a single belt slaved to force sensors appropriately positioned relative to a rigid part.

This analysis of the prior art shows that the various solutions enable either analysis of walking action or analysis of running action and always require, for each of the applications, independent apparatus which has consequences in terms of costs, overall size, etc.

The invention has set itself the task of overcoming these drawbacks in a simple, reliable, effective and efficient manner.

The problem that the invention aims to solve is to be able to analyse the ground reaction forces exerted during walking or running using a single apparatus.

In order to solve this problem, apparatus for measuring and analysing the forces exerted by the legs comprising a motor-driven treadmill slaved to two superposed assemblies mounted in combination with measuring sensors in order to analyse either running by means of the lower assembly or walking by means of both the lower assembly and the upper assembly as required has been developed.

In order to solve the problem of measuring either the ground reaction forces exerted during walking or the ground reaction forces exerted during running as required, the upper assembly comprises two independent platforms located in the same plane, one beside the other and in parallel, in order to match each of the legs, each platform being separately slaved to force sensors supported by a common platform constituting the lower assembly which is itself slaved to ground reaction force sensors.

Given the characteristics of the inventions, the motor-driven treadmill constitutes an endless belt that surrounds the two independent platforms and the common platform.

According to other aspects of the invention, given the problem of being able to measure and analyse various forces exerted during walking or running using a single apparatus, the sensors of the two platforms of the upper assembly may be of the unidirectional type in order to measure vertical forces whereas the sensors of the common platform are of the prestressed tridirectional type for measuring vertical, horizontal, posteroanterior and horizontal mediolateral forces.

Advantageously, the various sensors are located substantially in the four corners of the platforms in question which have a generally rectangular shape.

In one embodiment, the endless belt of the treadmill is slaved to a drive roller and a deflection roller mounted with a rotational drive capability on a support frame accommodating the common platform.

The invention is explained below in greater detail, reference being made to the accompanying drawings in which:

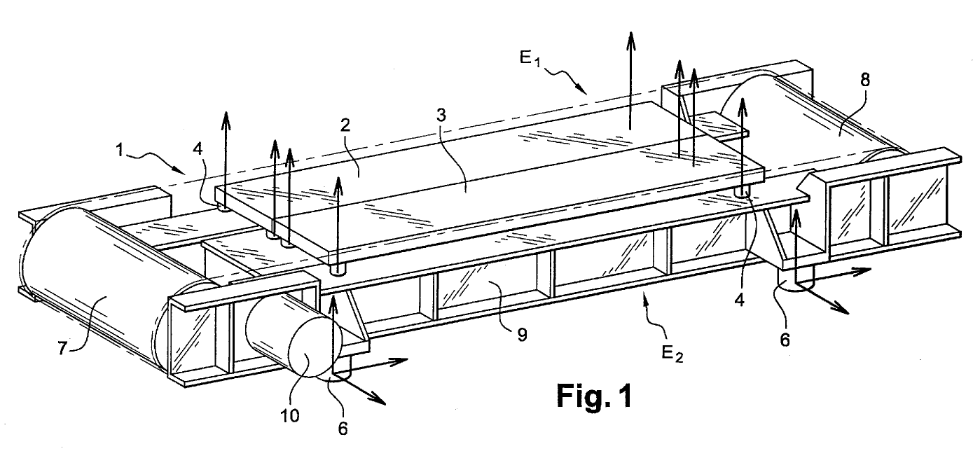

FIG. 1 is a schematic perspective view of one embodiment of the apparatus according to the invention;

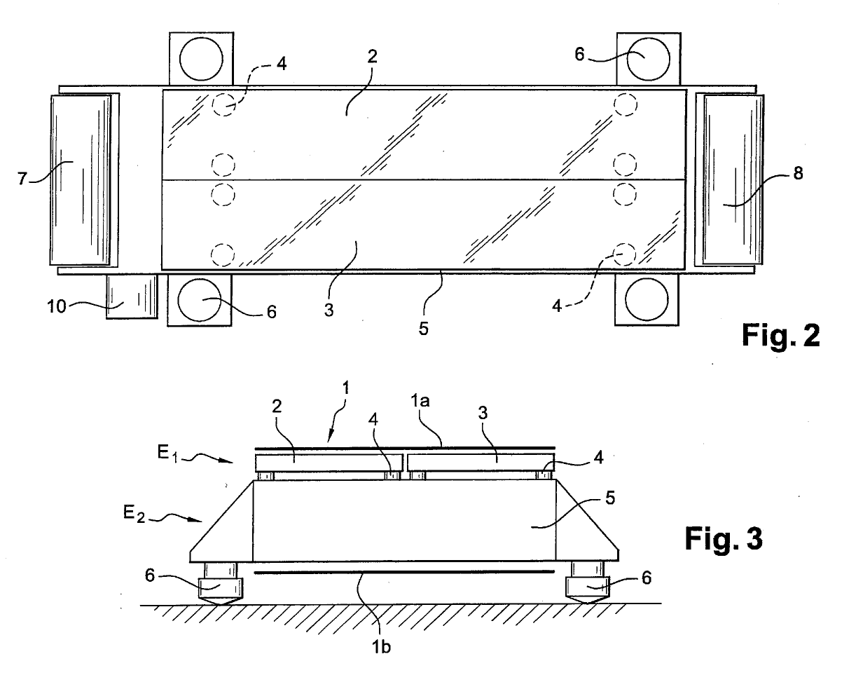

FIG. 2 is a schematic plan view showing the principle of the measuring and analysis apparatus;

FIG. 3 is a transverse cross-sectional view along line 3-3 in FIG. 2.

According to one basic aspect of the invention, the apparatus of the treadmill type comprises two superposed assemblies (E1) and (E2) for measuring and analysing forces exerted by the legs during either running or walking as required. As indicated in the rest of this description, the two assemblies, namely upper assembly (E1) and lower assembly (E2), are mounted in combination with endless belt (1) of the motor-driven treadmill. Similarly, the two measuring assemblies (E1) and (E2) are slaved to a central control unit in order to select analysis of walking or analysis of running.

Upper assembly (E1) comprises two independent rigid platforms (2) and (3) of generally rectangular shape. These two platforms (2) and (3) are located in the same plane, one beside the other and parallel, in order to match each of the legs. The gap between the two platforms (2) and (3) is extremely small so that said platforms (2) and (3) are located almost right up against each other whilst nevertheless being independent of each other. Each platform (2) and (3) which corresponds to each of the legs is independently slaved to force sensors (4).

Advantageously, for each of platforms (2) and (3), the sensors (4) are located substantially in the four corners. Each platform (2) and (3) therefore has four sensors (4). Sensors (4) are of the unidirectional type for measurements on a single axis corresponding to vertical forces. Use of multidirectional type sensors (4) is not excluded.

The various sensors (4) are supported by a rigid common platform (5) that constitutes lower measuring assembly (E2). This common platform (5) of generally rectangular shape is slaved to ground reaction force sensors (6) either directly or via a separately mounted flat surface. Sensors (6) which are located substantially in the four corners of common platform (5) are of the tridirectional (3 axes) type in order to measure vertical, horizontal, posteroanterior and horizontal mediolateral forces.

The endless belt (1) of the treadmill is slaved to a drive roller (7) and a deflection roller (8) mounted with a rotational drive capability on a support frame (9) that accommodates common platform (5). As shown in FIG. 3 in particular, the two strands (1a) and (1b) of the endless belt surround the two independent platforms (2) and (3) and common platform (5). Upper strand (1a) is located on the upper support surface of platforms (2) and (3) whereas lower strand (1b) is located under the lower surface of common platform (5).

Given the basic characteristics of the invention, it is apparent that lower assembly (E2) can be used to measure and analyse the reaction forces exerted during running when upper measuring assembly (E1) is not activated. Both the upper assembly (E1) and lower assembly (E2) are activated in order to measure and analyse forces exerted during walking.

The apparatus as defined, especially its frame (9), may have various accessories that are perfectly familiar to those skilled in the art, for example bars. Similarly, the apparatus may be designed to be used “off the ground” or in a pit. It should also be noticed that the various means of measurement and analysis are not described and are not part of the object of the invention and are perfectly familiar to those skilled in the art. Similarly, driving of roller (7), generally speaking by a motor (10), can be provided by various means.

The advantages of the invention are evident from the description, the possibility of using the treadmill to measure either pressure and ground reaction forces produced during walking or pressure and reaction forces produced during running whilst abiding by the constraints of the specific characteristics of analysis of walking and running as stated in the introductory part of the description is underlined and emphasised in particular.

1. Apparatus for measuring and analysing forces exerted by the legs comprising a motor-driven belt mounted above an upper assembly and a lower assembly mounted in combination with first measuring sensors and second measuring sensors in order to analyse at least one of running by means of lower assembly and walking by means of both lower assembly and upper assembly.

2. Apparatus as claimed in claim 1, wherein the upper assembly comprises two independent platforms located in the same plane, one beside each other and parallel, in order to match each of the legs, each of the two platforms being separately slaved to first force sensors supported by a common platform constituting lower assembly (E2), wherein the lower assembly is slaved to second force sensors.

3. Apparatus as claimed in claim 2, wherein motor-driven belt comprises endless belt surrounding the two independent platforms and the common platform.

4. Apparatus as claimed in claim 1, wherein the first measuring sensors of the two platforms of the upper assembly are capable of measuring vertical forces.

5. Apparatus as claimed in claim 1, wherein the second measuring sensors of the common platform are of the prestressed tridirectional type for measuring vertical, horizontal, posteroanterior and horizontal mediolateral forces.

6. Apparatus as claimed in claim 2, wherein the two platforms comprise rectangular platforms having four corners, and wherein the first measuring sensors and the second measuring sensors are located substantially in the four corners of the platforms.

7. Apparatus as claimed in claim 3, wherein the endless belt is slaved to a motor roller and a deflection roller mounted with a rotational drive capability on a support frame that accommodates common platform.

8. Apparatus as claimed in claim 2, wherein the first measuring sensors are capable of measuring vertical forces.

9. Apparatus as claimed in claim 3, wherein the first measuring sensors are capable of measuring vertical forces.

10. Apparatus as claimed in claim 2, wherein the second measuring sensors are of the prestressed tridirectional type for measuring vertical, horizontal, posteroanterior and horizontal mediolateral forces.

11. Apparatus as claimed in claim 3, wherein the second measuring sensors are of the prestressed tridirectional type for measuring vertical, horizontal, posteroanterior and horizontal mediolateral forces.

12. Apparatus as claimed in claim 3, wherein the two platforms comprise rectangular platforms having four corners, and wherein the first measuring sensors and the second measuring sensors are located substantially in the four corners of the platforms.

13. Apparatus as claimed in claim 4, wherein the two platforms comprise rectangular platforms having four comers, and wherein the first measuring sensors and the second measuring sensors are located substantially in the four corners of the platforms.

14. Apparatus as claimed in claim 5, wherein the two platforms comprise rectangular platforms having four corners, and wherein the first measuring sensors and the second measuring sensors are located substantially in the four corners of the platforms.
